(19) United States
(12) Patent Application Publication
(10) Pub. No.: US 2009/0028800 A1
(43) Pub. Date: Jan. 29, 2009

(54) Method of Treating Postpartum Depression and Aggression

(76) Inventors: William H. Peeples, Plantation, FL (US); Jerry T. Thornthwaite, Henderson, TN (US)

Correspondence Address: LANIER FORD SHAVER & PAYNE P.C., P O BOX 2087, HUNTSVILLE, AL, 35804-2087, US

(21) Appl. No.: 11/829,876

(22) Filed: Jul. 27, 2007

(51) Int. Cl. A61K 33/24; A61K 31/19; A61K 33/32; A61K 9/12; A61P 43/00

(52) U.S. Cl. 424/43; 424/617; 424/643; 514/557

(57) ABSTRACT

The present disclosure is directed towards administering a biologically effective amount of a thiomolybdate compound to a patient who demonstrates either depressive or aggressive behavior. According to the present disclosure, thiomolybdate compounds can be administered orally, sublingually, intravascular, intraperitoneal, intradermal, intramuscular, or via any other known administrative techniques. Administration of the thiomolybdate compound lowers the blood serum copper level in the patient, thereby reducing the efficacy of the copper levels which helped cause the onset or postpartum depression or aggression.

### FIELD OF THE DISCLOSURE

The present disclosure relates generally to health care. In particular, the present disclosure relates to a method of treating postpartum depression and aggression. More particularly, the present disclosure relates to administering copper reducing agents to treat postpardum depression and aggression.

### BACKGROUND OF THE DISCLOSURE

Depression affects millions of individuals worldwide each year. The causes of depression vary greatly from person to person, as do the prescribed treatment methods. While one single factor has not been singled out as the cause of depression, professionals have identified psychological, environmental and physiological factors which can trigger the onset of depression. Psychological factors such as grief, loss, financial problems, and divorce can often cause depression. Environmental factors such as poverty or abuse, contribute to the cause of depression, as do factors such as living with a depressed or ill family member. Physiological factors, often involving chemical imbalances in the body, can also cause depression. For example, thyroid conditions, hormone imbalance, and even viral infections have been linked to depression.

Treatment regimens for depression include psychotherapy, such as interpersonal therapy, cognitive-behavior therapy, and psychodynamic therapy; as well as medication such as selective serotonin reuptake inhibitors (SSRIs), tricyclic antidepressants (TCAs), and monoamine oxidase inhibitors (MAOIs). SSRIs, such as Paxil, Zoloft, and Prozac, affect the availability of the neurotransmitter serotonin, which has been linked to mood stabilization. Side effects associated with SSRIs, however, include nausea, headache, and tremor. TCAs, such as Elavil, Tofranil, and Pamelor, affect the availability of the neurotrasmitters norepinephrine, dopamine, and serotonin. However, TCAs also cause adverse side effects. MAOIs, such as Nardil and Parnate, also work to increase the levels of certain neurotransmitters in the brain. MAOIs can also cause a sharp increase in blood pressure, which makes them an unattractive treatment alternative.

Resent studies have shown that depression is also linked to high blood serum copper levels, possibly because copper can alter the balance of neurotransmitters in the brain. As an example, women with a history of postpartum depression tend to have high levels of copper in their blood. During pregnancy, blood serum copper levels more than double before normalizing after childbirth. Copper levels in women who develop postpartum depression, however, do not normalize. Accordingly, a method of normalizing blood copper levels is needed in order to prevent, treat, and control postpartum depression and other forms of depression linked to elevated blood serum copper levels.

Aggression is another disorder linked to high blood serum copper levels. Individuals prone to violence tend to have abnormal copper concentrations, again, possibly because copper can alter the balance of neurotransmitters in the brain. Although there have been some attempts to develop specific anti-aggression drugs, the current psychopharmacological treatment strategy involves the treatment of impulsivity. Individuals demonstrating impulsivity tend to make spur of the moment decisions that often result in a negative outcome. Impulsivity is evident in numerous psychiatric disorders, and many classes of medications have been prescribed for its treatment. These include the SSRIs, 5-HT receptor agonists and antagonists, lithium, anticonvulsants, antipsychotics, beta blockers, as well as alpha antagonists, opiate antagonists, and dopamine agonists. However, given the apparent role that blood serum copper plays in aggression, a method of normalizing or reducing blood copper levels is needed in order to prevent, treat, and control depression.

### SUMMARY OF THE DISCLOSURE

Applicant has addressed the need for effective treatment of depression and aggression by providing a method for reducing blood copper serum levels utilizing copper chelating compounds.

It is, therefore, a principle object of the subject disclosure to provide a method for reducing blood serum copper levels. More particularly, it is an object of the present disclosure to provide a method of administering a thiomolybdate which binds to blood serum copper, allowing for the expulsion of such copper from the body. In such context, it is still a more particular object of the present disclosure to provide the administration of tetrathiomolybdate.

Additional objects and advantages of the disclosure are set forth in, or will be apparent to those of ordinary skill in the art from, the detailed description as follows. Also, it should be further appreciated that modifications and variations to the specifically illustrated and discussed methods and compositions hereof may be practiced in various embodiments and uses of this disclosure without departing from the spirit and scope thereof, by virtue of present reference thereto. Such variations may include, but are not limited to, substitutions of the equivalent means, features, and compositions for those shown or discussed, and the functional or positional reversal of various parts, features, method steps, or the like.

Still further, it is to be understood that different embodiments of this disclosure may include various combinations or configurations of presently disclosed features, elements, method steps, or their equivalents, including combinations of features or configurations thereof not expressly stated in the detailed description. These and other features, aspects, and advantages of the present invention will become better understood with reference to the following descriptions and the appended claims.

### DETAILED DESCRIPTION

It will be apparent to those skilled in the art that various modifications and variations can be made in the present disclosure without departing from the scope and spirit of the disclosure as described herein. For instance, features illustrated or described as part of one embodiment can be included in another embodiment to yield a still further embodiment. Moreover, variations in selection of materials and/or characteristics may be practiced to satisfy particular desired user criteria. Thus, it is intended that the present disclosure covers such modifications as come within the scope of the present features and their equivalents.

Thiomolybdate is a known therapeutic composition which binds blood serum copper and forms an agent-copper-protein complex. Thiomolybdate compounds include dodecathiodimolybdate, tetrathiomolybdate, iron octathiodimolybdate, trithiomolybdate, dithiomolybdate, and monothiomolybdate. While thiomolybdate compounds have been used to treat diseases characterized by angiogenesis, such compounds are not known to be used to treat depression and aggression. Current treatments for these disorders often focus on neurotransmitter availability and manipulation rather than trace metal concentrations in blood serum. The present disclosure, therefore, utilizes thiomolydate compounds, as well as other copper chelating agents, such as penicillamide, to treat depression and aggression by lowering blood serum copper levels.

According to the present disclosure, a biologically effective amount of a thiomolybdate compound, e.g., tetrathiomolybdate, is administered to a patient who demonstrates either depressive or aggressive behavior. For purposes of this disclosure, a “biologically effective amount” is used to describe an amount effective to lower blood serum copper levels by any degree. In one embodiment, a biologically effective amount refers to an amount between 2 milligrams and 200 milligrams, inclusive, per patient per day.

Because thiomolybdate compounds slowly degrade when exposed to air, the proper storage and administration of such compounds are necessary. According to the present disclosure, thiomolybdate compounds could be administered orally, such as through encapsulation in liposomes, microparticles, microcapsules, capsules, via an inert gas in pill form (using “blister packs”), or sublingually. Furthermore, a biologically effective amount could be administered via intravascular, intraperitoneal, intradermal, intramuscular, or subcutaneous injection. In another embodiment, the compound could be administered via a nasal spray, or a subcutaneous vascular access infusion port which has been implanted in the patient. Various other known delivery systems may be used to administer thiomolybdate compounds such as intracerebral, intravaginal, transdermal, rectally, by inhalation, or topically. The preferred method of administration in a patient unable to take oral dosages is the use of intravenous delivery using drip bags.

In other embodiments, thiomolybdate compounds may be administered locally to the area in need of treatment by local infusion during surgery, topical application, by use of a catheter, suppository, implant (porous, non-porous, or gelatinous material, including membranes, such as sialastic membranes, or fibers. The thiomolybdate compound may be administered directly to the lung by inhalation using a Metered Dose Inhaler, a Dry Powder Inhaler (“DPI”), a multiple dose DPI, a liquid spray device supplied, a nebulizer, or an electrohydrodynamic aerosol device. In other embodiments, the thiomolybdate compounds can be delivered in a vesicle (liposome), or via sustained release systems such as those using polymeric materials, enteric-coated preparations osmotic delivery systems.

Administration of the thiomolybdate compound lowers the blood serum copper level in the patient, thereby reducing the efficacy of copper levels which caused the onset or postpartum depression or aggression. Once blood serum copper levels are normalized, copper levels can be maintained by the patient using a low copper diet. To compliment the effects of the reduced blood serum copper levels, current medications used to treat depression (SSRIs, TCAs, MAOIs) and aggression (SSRIs, 5-HT receptor agonists and antagonists, lithium, anticonvulsants, antipsychotics, beta blockers, alpha antagonists, opiate antagonists, and dopamine agonists) may be administered to the patient. To farther compliment the effects of the present disclosure, zinc levels may be increased. Therefore, a recommended oral dosage would include the administration of two twenty (20) milligram (mg) capsules of the thiomolybdate compound three (3) times per day with each meal, along with the administration of about thirty to fifty (30-50) milligrams (mg) of zinc three (3) times per day with each meal.

Although an embodiment of the disclosure has been described using specific terms and devices, such description is for illustrative purposes only. The words used are words of description rather than of limitation. It is to be understood that changes and variations may be made by those of ordinary skill in the art without departing from the spirit or the scope of the present disclosure, which is set forth in the following claims. In addition, it should be understood that aspects of various other embodiments may be interchanged both in part or in whole. Therefore, the spirit and scope of the appended claims should not be limited to the description of the preferred version contained herein.

We claim:

1. A method of preventing, treating, or controlling postpartum depression in a patient, which comprises administering to said patient a biologically effective amount of a first agent that binds or complexes copper.

2. The method of claim 1 wherein said first agent is a thiomolybdate.

3. The method of claim 2, wherein said thiomolybdate is tetrathiomolybdate.

4. The method of claim 1, wherein said biologically effective amount of said first agent is between about 2 milligrams and about 200 milligrams per day.

5. The method of claim 1, wherein said first agent is administered orally,

6. The method of claim 5, wherein said first agent is administered as an inert gas in pill form.

7. The method of claim 1, wherein said first agent is administered sublingual.

8. The method of claim 1, wherein said first agent is administered by intravascular injection.

9. The method of claim 1, where said first agent is administered by intramuscular injection.

10. The method of claim 1, where said first agent is administered by subcutaneous injection.

11. The method of claim 1, wherein said first agent is administered by a nasal spray.

12. The method of claim 1, wherein said first agent is administered via a subcutaneous vascular access infusion port which has been implanted in said patient.

13. The method of claim 1, wherein said first agent is administered using one of the group consisting of a Metered Dose Inhaler, a Dry Powder Inhaler (“DPI”), a multiple dose DPI, a liquid spray device supplied, a nebulizer, or an electrohydrodynamic aerosol device.

14. The method of claim 1, wherein said first agent is stored in an inert gas using blister packs of one or more capsules.

15. The method of claim 1, wherein said first agent is administered via sustained release systems selected from the group consisting of polymeric materials, enteric-coated preparations, or osmotic delivery systems.

16. The method of claim 1, further comprising the step of administering to the patient a therapeutically effective amount of at least a second agent.

17. The method of claim 16, wherein said second agent is selected from the group consisting of an SSRI, TCA, or MAOI.

18. The method of claim 1, further comprising the step of administering to the patient a therapeutically effective amount of zinc.

19. A method of preventing, treating, or controlling aggression in a patient, which comprises administering to said patient a biologically effective amount of a first agent that binds or complexes copper

20. The method of claim 19, wherein said first agent is a thiomolybdate.

21. The method of claim 20, wherein said thiomolybdate is tetrathiomolybdate.

22. The method of claim 19, wherein said biologically effective amount of said first agent is between about 2 milligrams and about 200 milligrams per day.

23. The method of claim 19, wherein said first agent is administered orally.

24. The method of claim 23, wherein said first agent is administered as an inert gas in pill form.

25. The method of claim 19, wherein said first agent is administered sublingual.

26. The method of claim 19, wherein said first agent is administered by intravascular injection.

27. The method of claim 19, where said first agent is administered by intramuscular injection.

28. The method of claim 19, where said first agent is administered by subcutaneous injection.

29. The method of claim 19, wherein said first agent is administered by a nasal spray.

30. The method of claim 19, wherein said first agent is administered via a subcutaneous vascular access infusion port which has been implanted in said patient.

31. The method of claim 19, wherein said first agent is administered using one of the group consisting of a Metered Dose Inhaler, a Dry Powder Inhaler (“DPI”), a multiple dose DPI, a liquid spray device supplied, a nebulizer, or an electrohydrodynamic aerosol device.

32. The method of claim 19, wherein said first agent is stored in an inert gas using blister packs of one or more capsules.

33. The method of claim 19, wherein said first agent is administered via sustained release systems selected from the group consisting of polymeric materials, enteric-coated preparations, or osmotic delivery systems.

34. The method of claim 19, further comprising the step of administering to the patient a therapeutically effective amount of zinc.
